(19) United States
(12) Patent Application Publication
(10) Pub. No.: US 2017/0182306 A1
(43) Pub. Date: Jun. 29, 2017

(54) Magnetic Medical Connector And Fluid Transfer Set Including The Magnetic Medical Connector

(71) Applicant: BAYER HEALTHCARE LLC, WHIPPANY, NJ (US)

(72) Inventor: MICHAEL J. SWANTNER, SAXONBURG, PA (US)

(21) Appl. No.: 15/123,062

(22) PCT Filed: Mar. 5, 2015
(86) PCT No.: PCT/US15/18898

§ 371 Date: Sep. 1, 2016

Related U.S. Application Data
(60) Provisional application No. 61/948,771, filed on Mar. 6, 2014.

(51) Int. Cl. A61M 39/24; F16K 31/08; A61M 39/10

(52) U.S. Cl. CPC A61M 39/24; A61M 39/10; F16K 31/084; A61M 5/007

(57) ABSTRACT

A connector for a fluid path set for delivery of a fluid to a patient during a procedure is described. The connector includes a magnetic check valve for limiting fluid flow to one direction through the fluid path. The magnetic check valve is configured to open in response to one or more of fluid pressure and change in value of magnetic force in the check valve.

### CROSS REFERENCE TO RELATED APPLICATIONS

This Application claims priority to U.S. Provisional Application 61/948,771, filed Mar. 6, 2014, the disclosure of which is incorporated herein by this reference. This disclosure also incorporates by reference U.S. Pat. No. 8,540,698 to Spohn et al.

### FIELD OF THE INVENTION

The present disclosure relates generally to connectors and check valves for fluid delivery systems for supplying fluids during medical diagnostic and therapeutic procedures and, further, to fluid transfer sets and flow controlling and regulating devices associated therewith used with fluid delivery systems.

### BACKGROUND

In many medical diagnostic and therapeutic procedures, a physician or other person injects a patient with a fluid. In recent years, a number of injector-actuated syringes and powered injectors for pressurized injection of fluids, such as contrast media, have been developed for use in procedures such as angiography, computed tomography, ultrasound, and NMR/MRI. In general, these powered injectors are designed to deliver a preset amount of contrast media at a preset flow rate.

Angiography is used generally in the detection and treatment of abnormalities or restrictions in blood vessels. In an angiographic procedure, a radiographic image of vascular structure is obtained through the use of a radiographic contrast medium, sometimes referred to simply as contrast, injected through a catheter. The vascular structures in fluid connection with the vein or artery in which the contrast is injected are filled with contrast. X-rays passing through the region of interest are absorbed by the contrast, causing a radiographic outline or image of blood vessels containing the contrast. The resulting images can be displayed on, for example, a monitor and recorded.

In a typical angiographic procedure, a physician places a cardiac catheter into a vein or artery. The catheter is connected to either a manual or to an automatic contrast injection mechanism. Automatic contrast injection mechanisms typically include a syringe connected to a powered injector having, for example, a powered linear actuator. Typically, an operator enters settings into an electronic control system of the powered injector for a fixed volume of contrast material and a fixed rate of injection. In many systems, there is no interactive control between the operator and the powered injector, except to start or stop the injection. A change in flow rate in such systems occurs by stopping the machine and resetting the parameters. Automation of angiographic procedures using powered injectors is discussed, for example, in U.S. Pat. Nos. 5,460,609, 5,573,515 and 5,800,397.

U.S. Pat. No. 5,800,397 discloses an angiographic injector system having high pressure and low pressure systems. The high pressure system includes a motor-driven injector pump to deliver radiographic contrast material under high pressure to a catheter. The low pressure system includes, among other things, a pressure transducer to measure blood pressure and a pump to deliver a saline solution to the patient as well as to aspirate waste fluid. A manifold is connected to the syringe pump, the low pressure system, and the patient catheter. A flow valve associated with the manifold is normally maintained in a first state connecting the low pressure system to the catheter through the manifold, and disconnecting the high pressure system from the catheter and the low pressure system. When pressure from the syringe pump reaches a predetermined and set level, the valve switches to a second state connecting the high pressure system/syringe pump to the catheter, while disconnecting the low pressure system from the catheter and from the high pressure system. In this manner, the pressure transducer is protected from high pressures, (see column 3, lines 20-37 of U.S. Pat. No. 5,800,397). However, compliance in the system components, for example, expansion of the syringe, tubing, and other components under pressure, using such a manifold system can lead to a less than optimal injection bolus. Moreover, the arrangement of the system components of U.S. Pat. No. 5,800,397 results in relatively large amounts of wasted contrast and/or undesirable injection of an excessive amount of contrast when the low pressure, typical saline system, is used. The injector system of U.S. Pat. No. 5,800,397 also includes a handheld remote control connected to a console. The control includes saline push button switches and a flow rate control lever or trigger. By progressive squeezing of the control trigger, the user provides a command signal to the console to provide a continuously variable injection rate corresponding to the degree of depression of the control trigger.

While manual and automated injectors are known in the medical field, a need generally exists for improved fluid delivery systems adapted for use in medical diagnostic and therapeutic procedures where fluids are supplied to a patient during the procedure. Additionally, a need generally exists for fluid transfer sets and flow controlling and regulating devices associated therewith that may be used with fluid delivery systems for conducting and regulating fluids flows. Moreover, a continuing need exists in the medical field to generally improve upon known medical devices and systems used to supply fluids to patients during medical procedures such as angiography, computed tomography, ultrasound, and NMR/MRI.

### BRIEF SUMMARY

The present disclosure is directed to a fluid delivery system comprising a fluid path set for use in the fluid delivery system. The fluid path set may comprise a connector member defining a lumen for fluid flow through the connector member and comprising a luer member in fluid connection with the lumen. A check valve arrangement may be disposed in the lumen of the connector member. The check valve arrangement may be configured to limit fluid flow to one direction through the connector member. The check valve arrangement comprises a magnetic element, such as an overmolded magnetic element, disposed in the lumen of the connector member and a retaining sleeve disposed in the lumen of the connector member. The retaining sleeve defines a central bore and comprises a distal end wall against which the overmolded magnetic element is adapted to magnetically seat to prevent fluid flow through a fluid flow aperture defined in the distal end wall and in the lumen until the overmolded magnetic element is dislodged from the distal end wall, for example, due to the fluid pressure within the central bore of the retaining sleeve and/or due to a change in magnetic attraction seating the overmolded magnetic element. In certain embodiments, the connector may comprise a magnetic element adapted to form a magnetic attractive bond to the overmolded magnetic element. In other embodiments, the connector may comprise a magnetic element adapted to form a magnetic repulsion to the overmolded magnetic element. Either the magnetic attractive force or the magnetic repulsive force may seat the overmolded magnetic element against the distal end wall.

Other embodiments of the present disclosure are directed to a connector for a fluid path set. The connector comprises a connector member defining a lumen for fluid flow through the connector member and a magnetic check valve arrangement disposed in the lumen of the connector member. The check valve arrangement comprises a magnetic element, such as an overmolded magnetic element, disposed in the lumen of the connector member and a retaining sleeve disposed in the lumen of the connector member. The retaining sleeve defines a central bore and comprises a distal end wall against which the overmolded magnetic element is adapted to magnetically seat to prevent fluid flow through a fluid flow aperture defined in the distal end wall and in the lumen until the overmolded magnetic element is dislodged from the distal end wall, for example, due to the fluid pressure within the central bore of the retaining sleeve and/or due to a change in magnetic attraction seating the overmolded magnetic element. In certain embodiments, the connector may comprise a magnetic element adapted to form a magnetic attractive bond to the overmolded magnetic element. In other embodiments, the connector may comprise a magnetic element adapted to form a magnetic repulsion to the overmolded magnetic element. Either the magnetic attractive force or the magnetic repulsive force may seat the overmolded magnetic element against the distal end wall.

A further embodiment of the present disclosure provides a method for reversibly sealing a valve of a fluid delivery system reactive to a specified pressure. The method comprises forming a magnetic attractive bond between an overmolded magnetic element and a distal end wall of a retaining sleeve disposed within a lumen of a connector member, wherein the overmolded magnetic element is seated over and prevents fluid flow through a fluid flow aperture defined in the distal end wall and wherein the magnetic attractive bond has a magnetic attractive bond strength equal to a specified pressure of a fluid within the lumen. The method may further comprise flowing a pressurized fluid through the lumen, wherein the fluid has a pressure greater than or equal to the specified pressure and dislodging the overmolded magnetic element from the distal end wall, thereby allowing fluid flow through the fluid flow aperture.

Other details and advantages will become clear when reading the following detailed description in conjunction with the accompanying drawings.

### BRIEF DESCRIPTION OF THE DRAWINGS

FIG. 1 is a front perspective view of an improved embodiment of the first connector member for use in a fluid path set, showing the first connector member incorporating a magnetic check valve arrangement.

FIG. 2 is a rear perspective view of the first connector member shown in FIG. 1.

FIG. 3 is a cross-sectional perspective view of the first connector member shown in FIGS. 1-2.

FIG. 4 is an enlarged cross-sectional perspective view of the first connector member shown in FIGS. 1-3 showing operational features thereof.

FIG. 5 is an exploded perspective view of the first connector member shown in FIG. 1

FIG. 6 is a front view of the first connector member shown in FIG. 1.

FIG. 7 is a cross-sectional view taken alone line A-A in FIG. 6.

FIG. 8 is a cross-sectional view taken alone line C-C in FIG. 6.

FIG. 9 is a detail view of Detail A in FIG. 7.

FIG. 10 is an exterior side view of the first connector member shown in FIG. 1.

FIG. 11 is a second exterior side view of the first connector member shown in FIG. 1.

FIG. 12 is a rear view of the first connector member shown in FIG. 1 with the check valve arrangement removed for clarity.

FIG. 13 is a detail view of Detail Z in FIG. 12.

FIG. 14 is a cross-sectional view of the first connector member shown in FIG. 1 along another longitudinal axis with overmolded magnetic element and retaining sleeve removed for clarity.

FIG. 15 is a perspective view of overmolded magnetic element used in the first connector member shown in FIG. 3.

FIG. 16 is a perspective view of a retaining sleeve used in the first connector member shown in FIG. 3.

FIG. 17 is a perspective view of the retaining sleeve viewed from the opposite end compared to FIG. 16.

FIG. 18 is a cross-sectional view of the retaining sleeve.

FIG. 19 is an exploded perspective view of an embodiment of a first connector member of U.S. Pat. No. 8,540,698 for use in the fluid path set of FIG. 10 of U.S. Pat. No. 8,540,698, showing the first connector member incorporating a check valve arrangement.

FIG. 20 is a longitudinal cross sectional view of the first connector member of FIG. 19.

FIG. 21 is a longitudinal cross sectional view of a second connector member adapted to connect to the first connector member of FIG. 19.

FIG. 22 is a longitudinal cross sectional view showing the first and second connector members of FIGS. 20 and 21.

FIG. 23 is a longitudinal cross sectional view of the first connector member of FIG. 19 in the form of a swivel-type first connector member.

FIG. 24 is an exploded perspective view of the swiveling first connector member of FIG. 23.

FIG. 25 is a cross sectional view taken along line 25-25 in FIG. 20.

FIG. 26 is a longitudinal cross sectional view of the first connector member of FIG. 20 having the check valve arrangement removed.

FIG. 27 is a longitudinal cross sectional view showing the first and second connector members connected as depicted in FIG. 22 and showing the results of fluid pressure acting on the check valve arrangement.

FIG. 28 is a cross sectional view taken along line 28-28 in FIG. 27.

### DETAILED DESCRIPTION

According to certain embodiments, the present disclosure provides for a connector and fluid path set for use in a fluid delivery system. The connector may be part of the fluid path set and may comprise a connector member defining a lumen for fluid flow through the connector member, wherein the connector member comprises a luer member in fluid connection with the lumen, and a check valve arrangement disposed in the lumen of the connector member, wherein the check valve arrangement is configured to limit fluid flow to one direction through the connector member. According to various embodiments, the check valve arrangement may be a magnetic check valve arrangement and comprise an overmolded magnetic element disposed in the lumen of the connector member, and a retaining sleeve disposed in the lumen of the connector member. The retaining sleeve may define a central bore and comprise a distal end wall against which the overmolded magnetic element is adapted to seat to prevent fluid flow through a fluid flow aperture defined in the distal end wall until the overmolded magnetic element is dislodged from the distal end wall. The overmolded magnetic element may be of any shape suitable to seat and seal against the distal end wall and seal the fluid flow aperture, for example a cylindrical shape, a conical shape, an ellipsoidal shape or a spherical shape. The presence of the overmolded magnetic element in the lumen, either seated to the distal end of the retaining wall by a magnetic force or pressed against the fluid flow aperture by a reverse fluid flow prevents fluid flow through the aperture is a retrograde direction, thereby making the magnetic check valve a one-way valve.

The overmolded magnetic element may comprise a magnetically active metal, as described herein, including a unitary magnetic element or a plurality of magnetic elements within a matrix. Alternatively, the overmolded magnetic element may comprise an overmolded metal element that is subject to magnetic attraction, such as iron or iron based alloys. Alternatively, the distal end wall may comprise a metal element subject to magnetic attraction. The magnetic attractive force described in relation to the various embodiments of the overmolded magnetic element and the magnetic element at the distal end wall of the retaining member may be a magnet-magnet attraction between a north-pole end of one magnetic element and a south-pole end of a second magnetic element, or may be a magnet-metal attraction between a magnetic element and a metal subject to magnetic attraction, e.g., a magnet in the overmolded magnetic element and a metal in the distal end wall or vice versa.

According to certain embodiments, the connector may further comprise a magnetic element at the distal end wall of the retaining sleeve, wherein the magnetic element is adapted to form a magnetic attractive bond to the overmolded magnetic element. In certain embodiments, the magnetic element may be a magnetically active metal located in the distal end wall of the retaining sleeve or located in the circumferential wall of the retaining sleeve or the connector member, such that it forms a magnetic attractive bond to the overmolded magnetic element to seat the overmolded magnetic element against the distal end wall of the retaining sleeve. Suitable magnetically active metals include, but are not limited to ferromagnetic materials, including iron, cobalt, nickel, gadolinium and dysprosium based ferromagnets, alnico magnets and rare earth magnets. The magnetically active metals may be a unitary magnetic element or may be a plurality of magnetic elements, for example, suspended in a matrix such as a polymeric matrix. According to these embodiments, the overmolded magnetic element is magnetically attracted to and seated against the distal end of the retaining member to seal and substantially prevent fluid flow through a fluid flow aperture. The magnetically attractive force between the overmolded magnetic element and the magnetic element has a value equal to a minimum pressure force, i.e., the crack pressure, required to dislodge or unseat the overmolded magnetic element from the distal end of the retaining sleeve, thereby allowing fluid flow through the fluid flow aperture. The overmolded magnetic element is generally dislodged from the fluid flow aperture when the pressure of the fluid in the central bore of the retaining sleeve has a pressure equal to or greater than the crack pressure.

According to other embodiments, the magnetic element may be an electromagnetic element generally located at the distal end wall of the retaining sleeve. The electromagnetic element may form a magnetic attractive bond with the overmolded magnetic element upon application of an electrical current to the electromagnetic element. According to these embodiments, the overmolded magnetic element is magnetically attracted to and seated against the distal end of the retaining sleeve by the electromagnetic element to seal and substantially prevent fluid flow through a fluid flow aperture. The magnetically attractive force between the overmolded magnetic element and the magnetic element has a value equal to a minimum pressure force, i.e., the crack pressure. According to various embodiments, the magnetically attractive force between the overmolded magnetic element and the electromagnet may be varied by varying the current flowing through the electromagnet. For example, in specific embodiments, the electromagnetic element may comprise a conductive wire coiled within or around at least one of the distal end wall of the retaining sleeve, a circumferential wall of the retaining sleeve, a circumferential wall of the connector member surrounding at least a portion of the retaining sleeve, a wall of the fluid path retaining element configured for holding the connector, and combinations of any thereof. A fluid path retaining element may be an element outside the fluid path, for example attached to a portion of a fluid injection system or a fluid injector, into which the fluid path and connector may be removably placed to secure the fluid path or connector at a specific location where the electromagnetic element may act upon the magnetic check valve. The coiled conductive wire may be in electrical communication with a source of electrical current, wherein the current may be either at a fixed voltage or current or at a variable voltage or current. The overmolded magnetic element may be seated against the distal end of the retaining element and seal the fluid flow aperture upon application of an electrical current to the electromagnetic element. Further, the overmolded magnetic element may be dislodged from and allow fluid flow through the fluid flow aperture by at least one of the pressure of a pressurized fluid in the central bore of the retaining sleeve having a pressure equal to or greater than the crack pressure of the magnetic check valve, reducing the electrical current applied to the electromagnetic element so that the crack pressure is reduced to less than the fluid pressure in the central bore, reversing the electrical current applied to the electromagnetic element, stopping the electrical current to the electromagnetic element, and combinations of any thereof. In other embodiments, the magnetic element may be a combination of a magnetically active metal and an electromagnetic element, which may work together to reversibly retain the overmolded magnetic element against the distal end of the retaining wall.

Still other embodiments of the magnetic check valve may use a magnetic repulsive force, formed between two like poles of the magnetic elements, to reversibly seat the overmolded magnetic element against the distal end wall of the retaining sleeve. For example, certain embodiments of the connector element may include an arrest located within the lumen generally opposite the distal end wall and distal to the overmolded magnetic element and configured to retain and arrest the overmolded magnetic element, and maintain it within proximity of the distal end wall of the retaining sleeve when it is laterally dislodged from the distal end wall. In specific embodiments, the arrest may comprise an arrest magnetic element oriented to produce a magnetic repulsive force between the arrest magnetic element and the overmolded magnetic element, e.g., by having a north-pole to north-pole interaction or south-pole to south-pole interaction between the arrest magnetic element and the distal end of the overmolded magnetic element. The magnetically repulsive force may force the overmolded magnetic element to seat against the distal end wall of the retaining element and seal the fluid flow aperture.

According to certain embodiments, the arrest magnetic element may be a magnetically active metal, such as described herein. The interaction between the arrest magnetic element and the overmolded magnetic element may be a magnet-magnet repulsive force that forces the overmolded magnetic element to seat against the distal end wall with a force equal to the crack pressure, such that a fluid pressure greater than the magnetic repulsive force, i.e., the crack pressure, dislodges the overmolded magnetic element from the distal end wall of the retaining wall, thereby unsealing the fluid flow aperture and allowing fluid flow therethrough.

According to other embodiments, the arrest magnetic element may be an electromagnetic element, such as a coiled conductive wire, located within the arrest element, within a circumferential wall of the connector member surrounding at least a portion of the arrest element, coiled around the outside of the circumferential wall of the connector member surrounding at least a portion of the arrest element within or around a wall of a fluid path retaining element configured for holding the connector, and combinations of any thereof. The coiled conductive wire may be in electrical communication with a source of an electrical current. The electromagnetic element of the arrest magnetic element may form a magnetic repulsive force against the overmolded magnetic element upon application of an electrical current to the electromagnetic element, thereby forcing the overmolded magnetic element to seat against the distal end wall of the retaining element. The overmolded magnetic element may be dislodged from the fluid flow aperture by at least one of a pressurized fluid having a pressure within the central bore of the retaining sleeve equal to or greater than the crack pressure (i.e., the magnetic repulsive force), reducing the electrical current applied to the electromagnetic element, reversing the electrical current applied to the electromagnetic element, stopping the electrical current applied to the electromagnetic element, and combinations thereof.

Other embodiments of the present disclosure include to a connector for a fluid path set. The connector comprises a connector member defining a lumen for fluid flow through the connector member and a magnetic check valve arrangement disposed in the lumen of the connector member. The magnetic check valve arrangement comprises a magnetic element, as described herein.

Still other embodiments of the present disclosure include a method for reversibly sealing a valve of a fluid delivery system or medical connector, wherein the valve is reactive to a specified pressure, such as a crack pressure. The valve may be a one-way crack valve, such as a one-way magnetic check valve as described herein. The method comprises forming a magnetic attractive bond between an overmolded magnetic element and a distal end wall of a retaining sleeve disposed within a lumen of a connector element, wherein the overmolded magnetic element is seated over and prevents fluid flow through a fluid flow aperture defined in the distal end wall and wherein the magnetic attractive bond has a magnetic attractive bond strength equal to a specified pressure of a fluid within the lumen. In specific embodiments, the method may further comprise flowing a pressurized fluid through the lumen, wherein the fluid has a pressure greater than or equal to the specified pressure, and dislodging the overmolded magnetic element from the distal end wall, thereby allowing fluid flow through the fluid flow aperture. According to other embodiments of the method the magnetic force may be a magnetic repulsive force between the overmolded magnetic element and a magnetic element within an arrest element distal to the overmolded magnetic element, wherein the magnetic repulsive force causes the overmolded magnetic element to be seated over and prevent fluid flow through a fluid flow aperture defined in the distal end wall and wherein the magnetic repulsive bond has a magnetic repulsive bond strength equal to a specified pressure of a fluid within the lumen, such that flowing a pressurized fluid through the lumen wherein the fluid has a pressure greater than or equal to the specified pressure dislodges the overmolded magnetic element from the distal end wall, thereby allowing fluid flow through the fluid flow aperture.

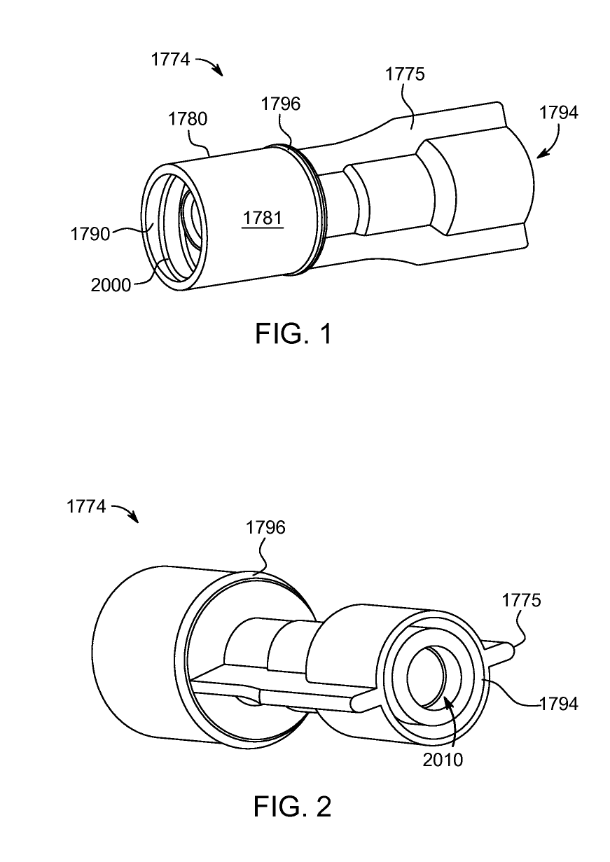

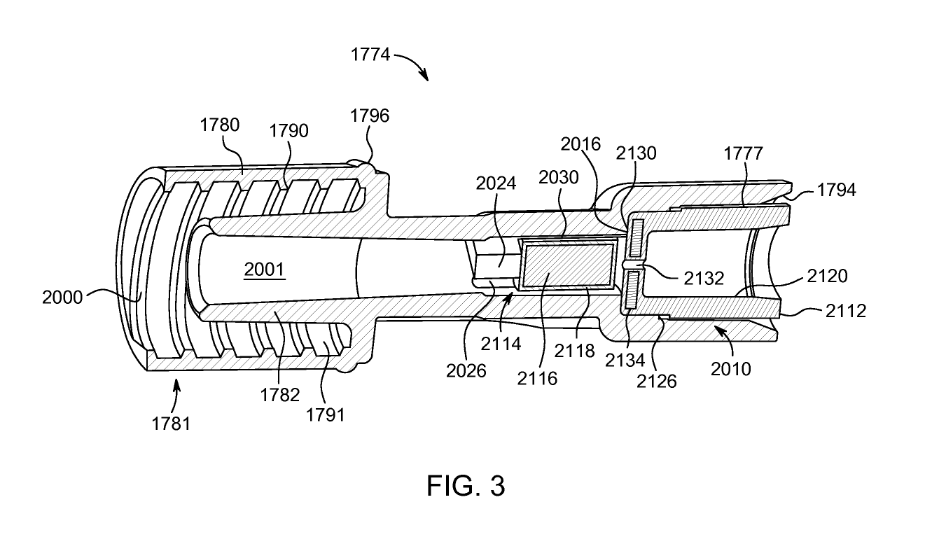

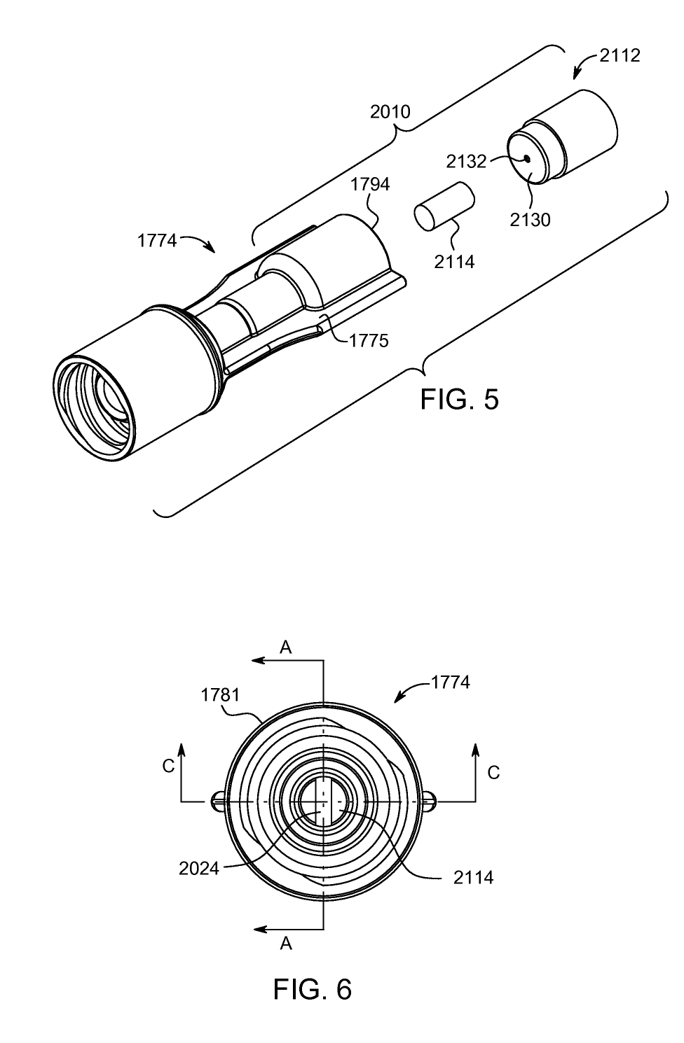

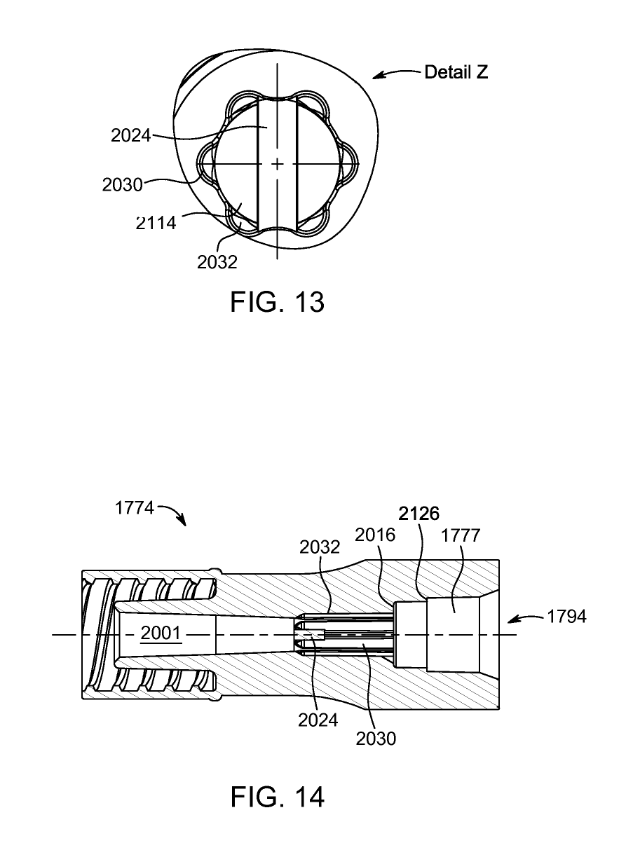

The various embodiments of the fluid path set and the magnetic check valve arrangement within the connector member will be better understood with reference to the following non-limiting figures. Referring to FIGS. 1-4, one embodiment of a fluid path set including a magnetic check valve according to the present disclosure may be achieved with a medical connector having a first connector member 1774 that comprises a magnetic check valve arrangement 2010 as illustrated in FIGS. 1-4. In this arrangement 2010, an overmolded magnetic element 2114 comprising permanent magnet or metal that is magnetically attractive 2116 that is encapsulated by an elastomer, for example a medical grade plastic overmolded around the magnetic element, to form a polymeric layer 2118 around the magnetic element 2116 and provide overmolded magnetic element 2114. The overmolded magnetic element 2114 is disposed within the fluid conducting cavity 2030. Cavity 2030 is desirably formed as a smooth bore cavity, although certain embodiments may include one or more grooves 2032 in the cavity walls (see FIGS. 12 and 13). As shown in FIGS. 1-4, the overmolded magnetic element 2114 is located in the fluid path 2001. The overmolded magnetic element 2114 is formed to have a north- and south pole and cylindrical shape to fit within the smooth bore cavity 2030 of connector member 1774. Other geometric shapes for overmolded magnetic element 2114 are also possible, for example, but not limited to a spherical, conical, or ellipsoidal shape, wherein the spherical or ellipsoidal overmolded magnetic element may act to seat against distal end wall 2016 and seal fluid flow aperture 2132. The polymeric layer 2118 exhibits an overmolded shape to form a seal on the opposing ends of the permanent magnet 2116. The overmolded magnetic element 2114 is housed inside of the first connector member 1774, which may be an injection molded body. The injection-molded body may be molded from any thermoplastic such as polycarbonate, for example clear polycarbonate or other polymeric material that is inert to the material flowing through the connector valve, and contains the overmolded magnetic element 2114. The permanent magnet 2116 allows fluid to pass through the area around the overmolded magnetic element 2114, with this area being generally annular-shaped to extend circumferentially around the overmolded magnetic element 2114. The overmolded magnetic element 2114 and its retaining sleeve or element (described herein) may be associated with the body of the first connector member 1774 by a variety of means, such as overmolding, assembly and UV adhesive, and trapping by another component which is bonded by ultrasonic laser welding. One suitable approach is to overmold the overmolded magnetic element 2114 and its retaining sleeve 2112 or element (discussed herein) into the body of the first connector member 1774 as the body is formed by an injection-molding process, as this approach can reduce concerns of biocompatibility and particulate contamination. Alternatively, overmolded magnetic element 2114 may be inserted into the retaining sleeve receiving cavity 1794 at the proximal end of the first connector member 1774. FIG. 5 illustrates one assembly process to insert overmolded magnetic element 2114 into receiving cavity 1794 followed by retaining sleeve 2112. Retaining sleeve 2112 may then be adhered or welded to the inner wall of receiving cavity 1794.

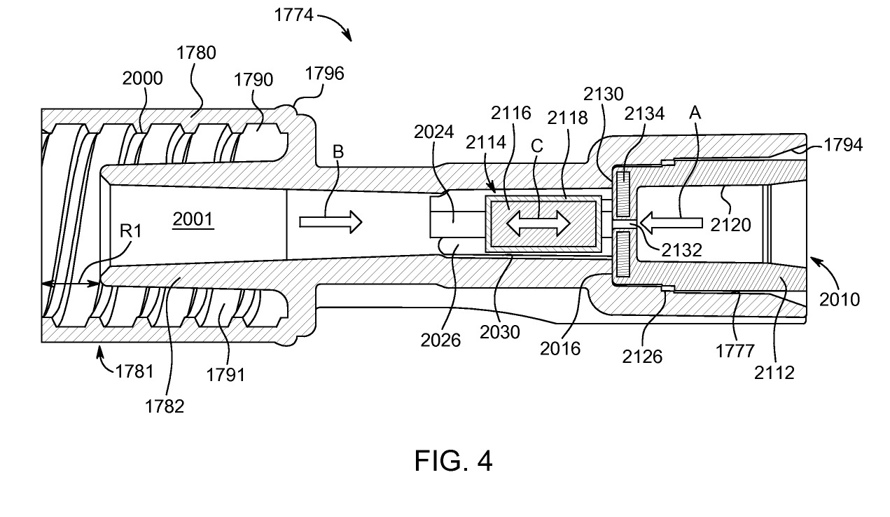

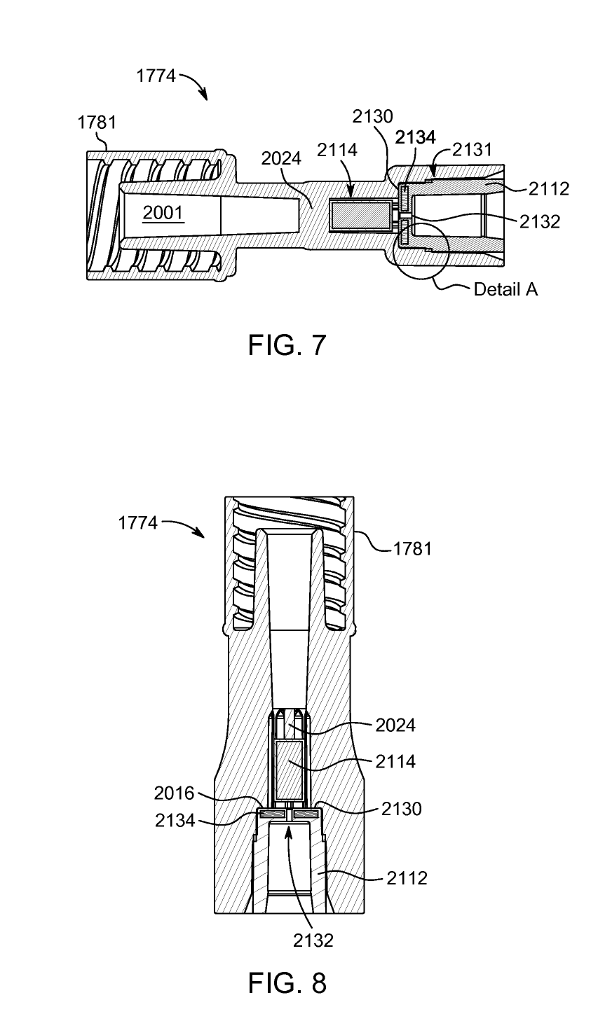

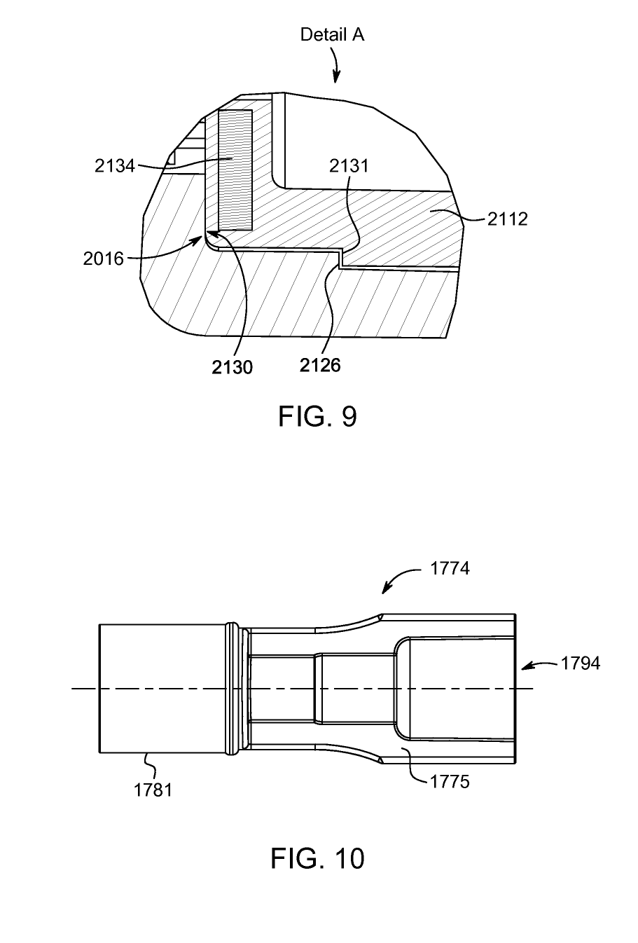

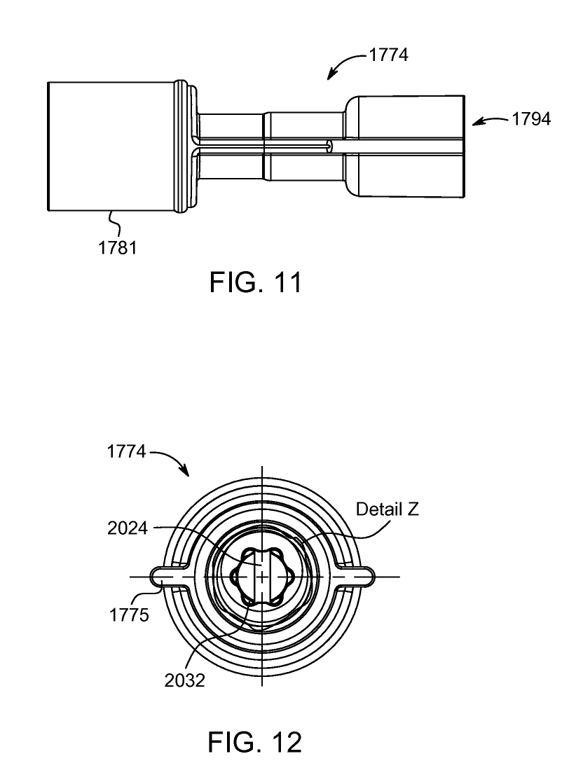

The hollow cylindrical retaining sleeve 2112 is seated within the conduit receiving cavity 1794 of lumen 1777 so that the retaining sleeve 2112 abuts the internal shoulder 2016 in the first connector member 1774. The conduit receiving cavity 1794 defines the internal shoulder 2016, and may define a second, proximal internal shoulder 2126 configured to abut complementary shoulder 2131 on retaining sleeve 2112. The retaining sleeve 2112 is shaped and disposed within the conduit receiving cavity 1794 of lumen 1777 so that the retaining sleeve 2112 abuts the shoulders 2016, 2126 (see Detail A in FIG. 9). The retaining sleeve 2112 may be formed of a medical grade polymer such as polycarbonate and like material and secured in the lumen by any suitable adhesive-joining technique, welding technique, or may be formed integrally with the body of the first connector member 1774. In certain embodiments, the retaining sleeve 2112 may be seated and disposed in the lumen 1777 during an overmolding process wherein the body of the first connector member 1774 is formed around the retaining sleeve 2112. FIGS. 4 and 8 illustrate a side view of connector member 1774 with overmolded magnetic element 2114 and retaining sleeve 2112 along line C-C of FIG. 6 and FIG. 7 illustrates a top view of connector member with overmolded magnetic element 2114 and retaining sleeve 2112 along line A-A of FIG. 6. FIGS. 10 and 11 illustrate exterior views of connector member 1774 along lines C-C and A-A of FIG. 6, respectively.

FIG. 12 illustrates an end-on view of the proximal end of the first connector member 1774 having the overmolded magnetic element 2114 and retaining sleeve 2112 removed for clarity. FIG. 12 shows arrest element 2024 configured for arresting distal movement of overmolded magnetic element 2114 upon unseating from distal end wall 2130. One or more grooves 2032 are shown on inner circumferential wall 2030 which allow improved flow of fluid around overmolded magnetic element 2114, once unseated from distal end wall of 2130. Wings 1775 configured for reversibly tightening and removing first connector member 1774 to a second complementary connector member (not shown) extend radially from the outer body of first connector member 1774. Detail Z of FIG. 12 is shown FIG. 13 clearly displaying one or more grooves 2032 and arrest 2024 of first connector member 1774. FIG. 14 displays a cross-sectional side-view of the first connector member 1774 along longitudinal axis with overmolded magnetic element and retaining sleeve removed for clarity. FIG. 14 shows elements of first connector member 1774 including arrest 2024, one or more grooves 2032, inner cavity 2030, and shoulders 2016 and 2126 in receiving cavity 1794 of lumen 1777.

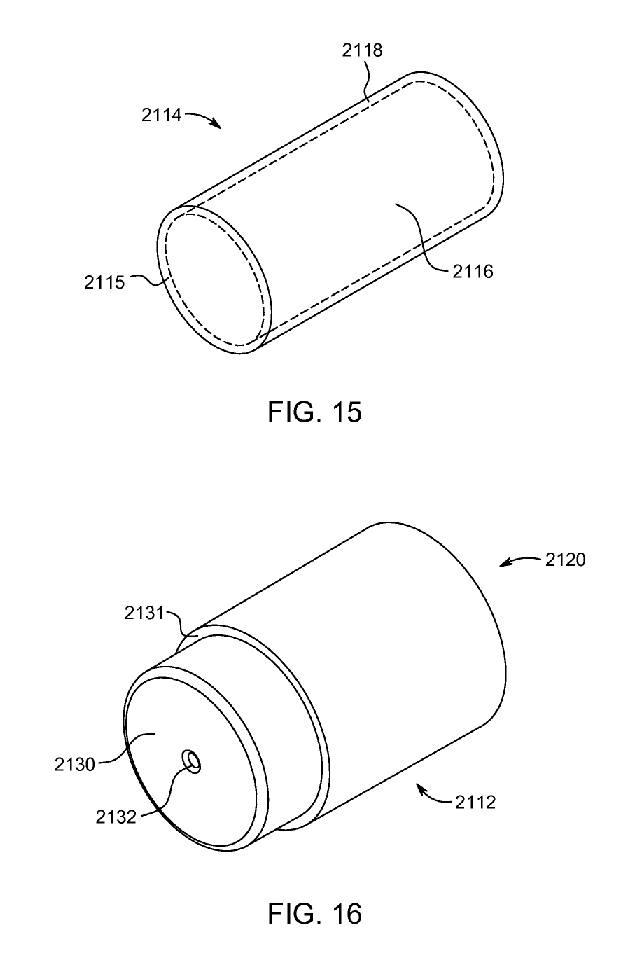

FIG. 15 displays a cylindrical overmolded magnetic element 2114 having substantially flat end 2115 configured for seating against distal end wall 2130 and sealing fluid flow aperture 2132 when fluid pressure is below the specific pressure. Other three dimensional shapes for overmolded magnetic element 2114 having an end configured for sealing fluid flow aperture 2132 are possible. For example, spheroidal or ellipsoidal overmolded magnetic elements may seal fluid flow aperture 2132 with a portion of an arced surface. Other shapes for overmolded magnetic element, such as shapes having protrusions which fit into fluid flow aperture 2132 and seal against the inner walls of the fluid flow path of fluid flow aperture 2132 are also envisioned. Magnetic element 2116 of overmolded magnetic element 2114 is displayed in dashed lines in FIG. 15. Magnetic element 2116 may be a ferromagnetic element configured for magnetic attraction of magnetic element 2134 of retaining sleeve 2112. In other embodiments, magnetic element 2116 may be a magnet attracted metal, such as iron ore or other metal, which is attracted to the magnetic field emanating from magnetic element 2134 of retaining sleeve 2112. Magnetic element 2116 of overmolded magnetic element 2114 is overmolded with a medical grade polymeric material 2118, as described herein. In other embodiments, magnetic element 2114 may be coated using other suitable coating methods, such as by a dip molding process to create a dip coated magnetic element, or by a coating process to create a coated magnetic element that are equivalent to and may be substituted for the overmolded magnetic element 2114 according to those embodiments.

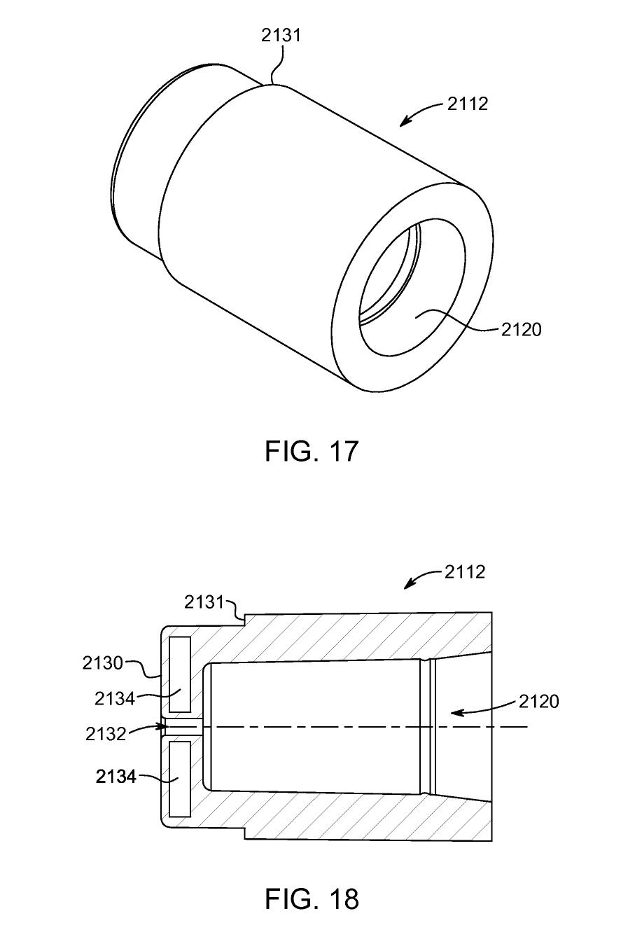

FIGS. 16-18 illustrate various views and elements of a retaining sleeve 2112 according to various embodiments. The retaining sleeve 2112 comprises a distal end wall 2130 defining a fluid flow aperture 2132 therein communicating with a central bore 2120 permitting fluid in the lumen 1777 to conduct through the sleeve 2112 and enter the cavity 2030. The distal end wall 2130 in this embodiment has a magnetic element 2134 (see e.g., FIG. 18), such as a magnetically attracted metal or a permanent magnet, as described herein, which magnetically attracts the magnetic element 2116 of overmolded magnetic element 2114. In another embodiment, magnetic element 2134 may comprise an electromagnet that may be reversibly toggled from an off state to an on state by application of an electric current to a coiled conductive wired in distal end wall 2130. According to these embodiments, application of an electric current to electromagnet 2134 may produce a magnetic field, attracting overmolded magnetic element 2114 to a seated, sealed position. Cutting or stopping the electric current to electromagnet 2134 eliminates the magnetic attraction between the electromagnet 2134 and overmolded magnetic element 2114, thereby releasing the seal and allowing fluid to flow. In this embodiment, when the electric current is cut, the crack pressure will be essentially zero. Further, reversing the electric current may convert the magnetic attractive force between overmolded magnetic element 2114 and electromagnetic element 2134 into a magnetic repulsive force causing overmolded magnetic element 2114 to move away from distal end wall 2130, as described herein. In specific embodiments, variation in current strength of the electric current may vary the electromagnetic strength of electromagnetic element 2134, thereby varying the strength of the magnetic attractive force between electromagnetic element 2134 and overmolded magnetic element 2114. According to this embodiment the crack pressure of the magnetic valve may be tuned or selected according to a desired crack pressure by selecting an electric current strength that provides the desired magnetic attractive force between electromagnetic element 2134 and overmolded magnetic element 2114. In yet other embodiments where distal end wall 2130 comprises a permanent magnet or an electromagnet, overmolded element 2114 may comprise an overmolded magnetically attractive metal, such as but not limited to an iron or an iron metal alloy, overmolded with an inert polymer layer 2118, wherein a magnetic attractive force between overmolded magnetically attractive metal element 2114 and the permanent magnet or electromagnet 2134 in distal end wall 2130 form a pressure active seal of fluid flow aperture 2132. Magnetic element 2134 may be attached to or within retaining sleeve 2112 by one of an overmolding process; or in other embodiments, the magnetic element 2134 may be bonded or attached to the surface of the distal end wall 2130 or in one or more depressions in the distal end wall 2130 by an adhesive, such as a UV cure adhesive, or by a snapfit to the surface or within the opening by one or more latch feature on distal end wall or within the depressions in the distal end wall 2130 of retaining sleeve 2112.

FIG. 4 illustrates schematically the operation of the check valve arrangement 2010. In use, when fluid is under a pressure greater than or equal to a specified pressure (or crack pressure) in lumen 1777 in the direction of arrow A, the fluid passes through the retaining sleeve 2112 and fluid flow aperture 2132 to act upon the overmolded magnetic element 2114. Once the pressure of the fluid reaches the specified pressure, the pressure causes the overmolded magnetic element 2114 to unseat from engagement with the distal end wall 2130 and permit fluid flow through the fluid flow aperture 2132, passing downstream around the overmolded magnetic element 2114, and past overmolded element arrest 2024 in the central opening 2022, which divides the central opening 2022 into two or more output channels 2026 and maintains the overmolded element 2114 in proximity to distal end wall 2130. The fluid flow passes through the cavity 2030 in the open annular space defined around the overmolded magnetic element 2114, for example through the one or more grooves 2032 (see FIG. 12). In other embodiments, the open annular space may not comprise one or more grooves 2032 and the fluid flows around the exterior of overmolded magnetic element 2114, for example when overmolded magnetic element 2114 has a radial diameter significantly less than the inner diameter of cavity 2030. When the fluid flow in the lumen 1777 is discontinued or fluid pressure falls below the specified pressure, the magnetic attraction between the permanent magnet or metal 2116 in overmolded magnetic element 2114 and the metal or magnetic element 2134 causes the overmolded magnetic element 2114 to reseat against the distal end wall 2130 and seal the fluid flow aperture 2132 to stop fluid flow through the magnetic check valve. Reverse fluid flow in the direction of arrow B is not possible with this embodiment of the check valve, since flow in direction B will cause the overmolded magnetic element 2114 to maintain the seat and seal against distal end wall 2130. Arrow C in FIG. 4 shows the bidirectional movement capability of the overmolded magnetic element 2114. The polymeric layer 2118 around the permanent magnet 2116 of overmolded magnetic element 2114 may seat against the distal end wall 2130 and seal around the fluid flow aperture 2132.

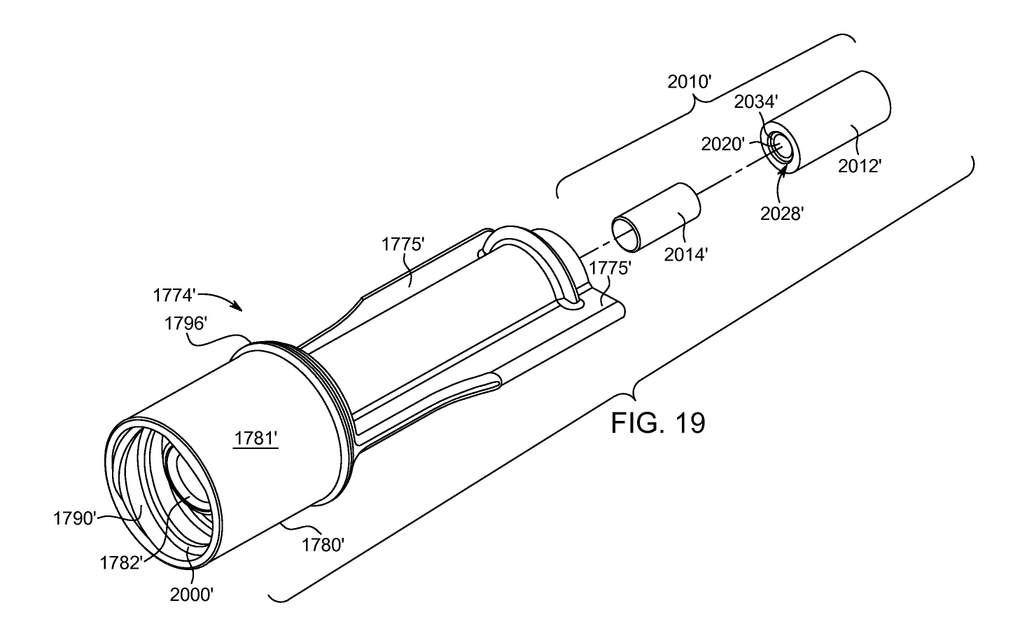

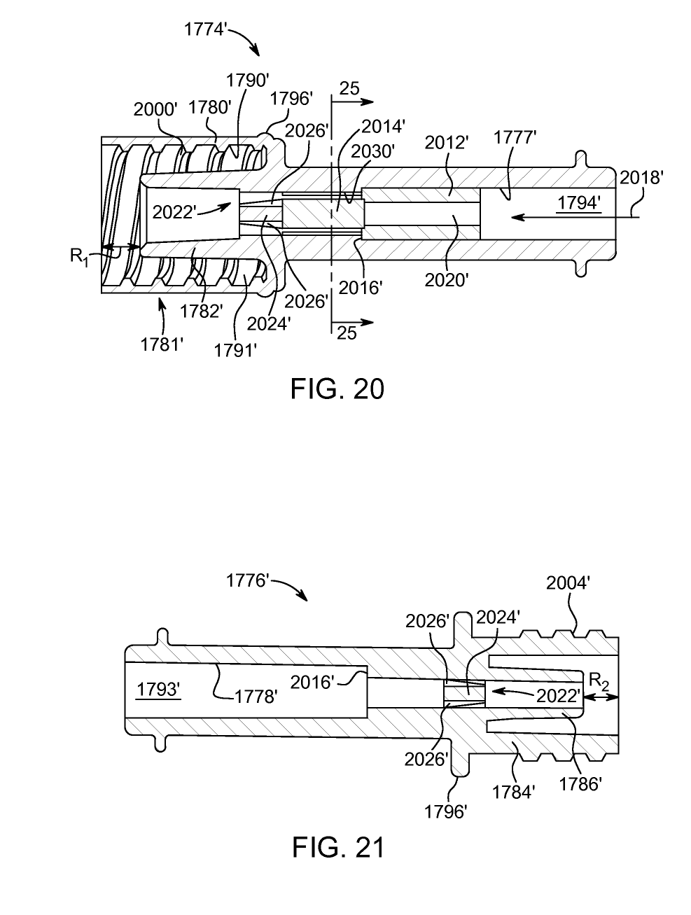

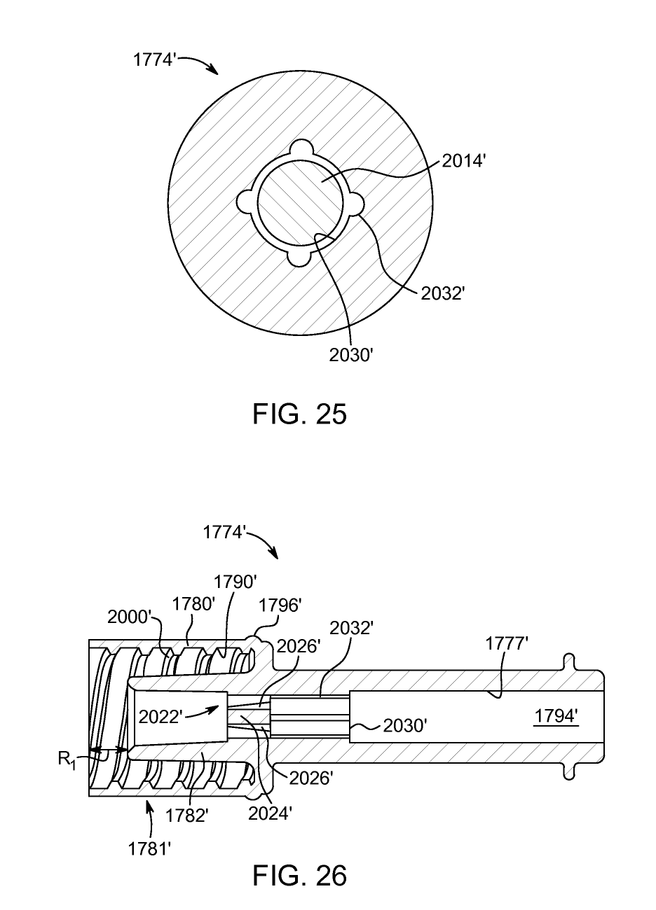

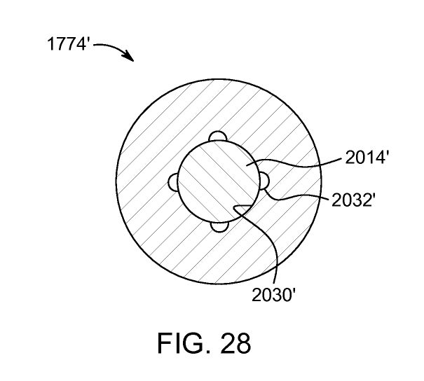

Referring to FIGS. 19-38, which reproduce FIGS. 37-46 from U.S. Pat. No. 8,540,698 to Spohn and describe an embodiment of a first connector member 1774′ for use in a medical connector 1708′ that contains a pressure active check valve that lacks magnetic elements, the figures will be utilized to describe structural features of the connector member common with the connector member comprising the magnetic check valve of the present disclosure. Features similar to those present the described medical connector with a magnetic check valve will have same numeric identifiers but will be differentiated with a prime mark (′) afterwards. The disclosure of U.S. Pat. No. 8,540,698 is incorporated in its entirety by this reference. Descriptions of features of the connector member of U.S. Pat. No. 8,540,698 which appear in the depicted embodiments of the present medical connector with magnetic check valve and/or do not conflict with the operation of the magnetic check valve according to the various embodiments described herein (such as, but not limited to, various features for connecting connector member 1774 to a second connector member or one or more fluid lines), will have substantially the same function and structure, except where necessarily different. The medical connector 1708′ is used to connect first and second sections in a fluid path set as depicted in FIG. 10 of U.S. Pat. No. 8,540,698. The medical connector 1708′ includes first and second connector members 1774′, 1776′.

The first connector member 1774′ is formed with an internally-threaded outer housing 1780′. The inner wall or surface 1790′ of the outer housing 1780′ defines internal threads 2000′. The outer surface 1781′ of the outer housing 1780′ may have a smooth texture as illustrated in FIG. 19, or include longitudinally-extending raised ribs 2002′ as illustrated in FIG. 24 to be discussed herein. Similar structural features may also occur on the outer housing of 1780 of the connector member 1774 comprising a magnetic check valve 2010, as described herein.

The first connector member 1774′ does not include external threads on this component. The “first member” 1782′ without external threads is formed substantially as a conventional female luer fitting, but is recessed a distance R1 within outer housing 1780′. This element may be referred to herein as the “first luer member 1782′”. The first luer member 1782′ and outer housing 1780′ define an annular cavity 1791′ therebetween for receiving the second threaded member 1784′ of the second connector member 1776′, which are likewise detailed in U.S. Pat. No. 8,540,698. As the outer housing 1780′ is disposed coaxially and concentrically about the first luer member 1782′, the outer housing 1780′ may be referred to as the “first annular member 1780”.

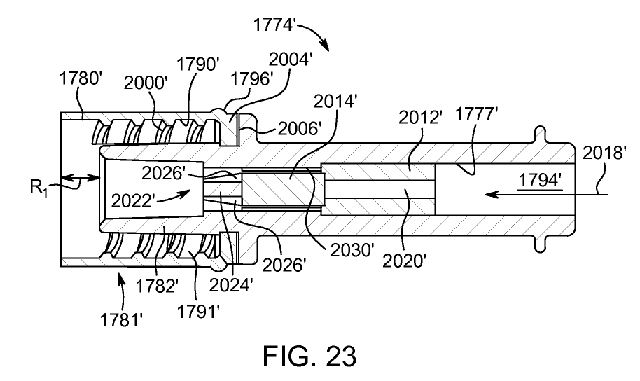

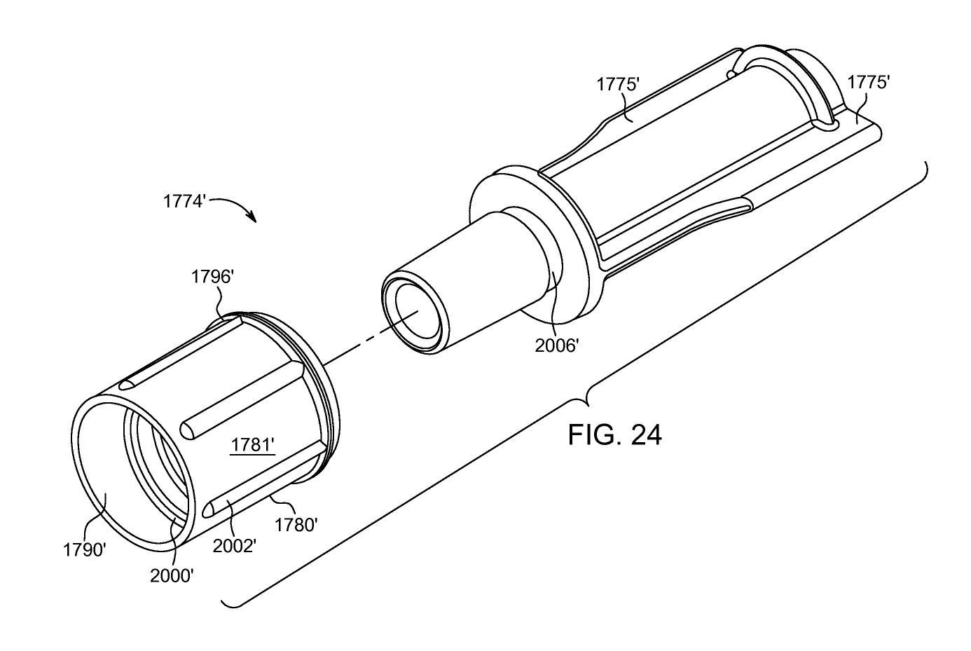

With specific reference to FIGS. 23 and 24, the outer housing or first annular member 1780′ may be adapted to rotate or “swivel” relative to the first luer member 1782′ in the first connector member 1774′ so that the connector 1708′ may be a “swiveling” connector. As shown in these two figures, the first annular member 1780′ includes an annular flange 2004′ that cooperates or engages a circumferentially extending recess 2006′ defined adjacent the first luer member 1782′. The flange 2004′ may rotationally slide in recess 2006′ so that the first annular member 1780′ may rotate or swivel relative to the first luer member 1782′. Similar swivel features may be incorporated into the connector member 1774 comprising a magnetic check valve 2010, as described herein.

The fluid path set illustrated in FIG. 10 of U.S. Pat. No. 8,540,698 includes two medical connectors 1708′ for connecting the first and second sections in the fluid path set. The rotational or swiveling feature of the first annular member 1780′ allows the first connector member 1774′ in each of the connectors 1708′ to be joined to the second connector member 1776′ in each of the connectors 1708′ without disturbing or altering the orientation of the respective input/output lines associated with the connectors 1708′ (see FIG. 10 of U.S. Pat. No. 8,540,698). For example, the connector 1708′ associated with the high pressure input/output lines connected to a syringe may be joined with the “swivel” connector 1708′ so that the orientation of a downstream pressure isolation mechanism is undisturbed. Thus, once the downstream orientation of the pressure isolation mechanism is set to a desired orientation by an operator of the fluid delivery system, the swiveling feature of the first connector member 1774′ may be used as a way of ensuring that this desired orientation is maintained. Without this swivel feature, it is possible that rotational force may be applied to the pressure isolation mechanism when the first and second connector members 1774′, 1776′ are joined in the two connectors used in the fluid path set, causing the pressure isolation mechanism to be rotated to an undesirable position. The swiveling feature ensures that rotational force is not substantially applied to the pressure isolation mechanism or fluid path thereby altering its orientation when the first and second section sections of the fluid path set are connected.

The first and second connector members used in the fluid path set may reverse locations for the first and second connector members 1774′, 1776′ so that the “high” pressure side of the first section of the fluid path set is not inadvertently connected to the “low” pressure side of the second section of the fluid path set and vice versa. The raised longitudinal ribs 2002′ on the outer housing 1780′ (see, e.g., FIG. 24) further improve the ability of the operator to make the connection between the first and second connector members 1774′, 1776′ by improving the frictional engagement between an operator's fingertips and the outer housing or first annular member 1780′ when rotating the first annular member 1780′ to threadedly engage the second threaded member 1784′ associated with the second connector member 1776′.

The second connector member 1776′ is adapted to threadedly engage the internal threads 2000′ provided on the inner surface 1790′ of the outer housing or first annular member 1780′. The second threaded member 1784′, which may be referred to as “second annular member 1784′” in an analogous manner to the first annular member 1780′, is now formed with external threads 2004′ on the external surface 1789′ of the second annular member 1784′ for engaging the internal threads 2000′ within the first annular member 1780′ of the first connector member 1774′. The external threads 2004′ threadedly engage the internal threads 2000′ within the first annular member 1780′ to connect the first and second connector members 1774′, 1776′.

In addition to securing the threaded engagement between the first and second connector members 1774′, 1776′, the external threads 2004′ form a tortuous path (not shown) or tortuous barrier for inhibiting or substantially preventing liquid flow out of or into liquid-trapping chamber 1792′. The tortuous path formed by the external threads 2004′ now acts to substantially prevent liquid flow rather than just inhibiting liquid flow. This result is because the engagement between the internal and external threads 2000′, 2004′ substantially closes off the liquid-trapping chamber 1792′ in a substantially liquid tight manner, substantially sealing off chamber 1792′.

The second connector member 1776′ also includes a recessed luer fitting or member 1786′, for example a male luer fitting, that is adapted to engage the first luer member 1782′ which, as indicated previously, may be formed as a female luer fitting. This “second” luer member 1786′ is recessed within the second annular member 1784′ by a distance R2. The first and second connector members 1774′, 1776′ are each adapted to receive a protector cap (see FIGS. 18 and 19 of U.S. Pat. No. 8,540,698).

According to specific embodiments, the first and second luer members 1782′, 1786′ are not required to be recessed within the first and second annular member 1780′, 1784′ and may extend substantially flush with the first and second annular members 1780′, 1784′. Additionally, in certain embodiments only one of the first and second luer members 1782′, 1786′ may be recessed within the first and second annular members 1780′, 1784′. For example, in certain embodiments the first luer member 1782′ may extend to be substantially flush with the first annular member 1780′ for increased positive locking engagement (i.e., increased surface area of engagement) with the second luer member 1786′. The first annular member 1780′ may provide a gripping surface for an operator's fingertips and will help ensure that contact is not made with the first luer member 1782′. In this situation, the second luer member 1786′ may be recessed as indicated previously. However, the second luer member 1786′ may be extended to be flush with the second annular member 1786′. In view of the foregoing, the first and second luer members 1782′, 1786′ may both be recessed or substantially flush with respect to the first and second annular members 1780′, 1784′, or only one of the first and second luer members 1782′, 1786′ may be recessed within the first and second annular members 1780′, 1784′ while the other is substantially flush with the first and second annular members 1780′, 1784′.

To join the first and second connector members 1774′, 1776′ together, the user inserts the second annular member 1784′ partially into first annular member 1780′ of the first connector member 1774′ until the external threads 2004′ on the second annular member 1784′ contact and begin to engage the internal threads 2000′ provided on the inner surface 1790′ of the first annular member 1780′. Once in position, the user may begin rotating the first annular member 1780′ so that the opposing external and internal threads 2004′, 2000′ associated with the second annular member 1784′ and first annular member 1780′, respectively, engage and draw the first and second connector members 1774′, 1776′ into threaded engagement. As the first and second connector members 1774′, 1776′ are drawn together, the second luer member 1786′, which is typically recessed within the second annular member 1784′, is received in the first luer member 1782′ thereby completing the fluid connection between lumens 1777′, 1778′. It will be understood that the present disclosure is intended to include a reversed configuration for the “male” second luer member 1786′ and “female” first luer member 1782′. In such a reversed configuration, the male second luer member 1786′ may be formed as a female luer fitting, and the first luer member 1782′ may be formed as a male luer fitting.

The connectors 1708′ used in the fluid path set may further include a check valve arrangement 2010′, including the magnetic check valve described herein, for limiting flow through the connectors 1708′. The check valve arrangement 2010′ may be disposed within lumen 1777′ of the first connector member 1774′, or within lumen 1778′ in the second connector member 1776′ depending on which direction through the connector 1708′ it is desired to limit flow.

The check valve arrangement 2010′ is provided in one or both of the connectors 1708′ used to connect the first proximal section to the second distal section of the fluid path set to isolate the first section from the second section unless pressure is present in the lines of the first proximal section.

The check valve arrangement 2010′ associated with the connectors 1708′ is normally closed until fluid pressure in the connectors 1708′ is sufficient to open the respective check valve arrangements 2010′ permitting flow through the connectors 1708′. Such pressure may be supplied, for example, by a peristaltic pump or other fluid pressurizing device associated with input line and a syringe associated with input line (see FIG. 10 of U.S. Pat. No. 8,540,698). For example, the connector 1708′ associated with input line may be configured such that the first connector member 1774′ of the connector 1708′ is associated with input line. The check valve arrangement 2010′ may be provided in the first connector member 1774′ to prevent secondary injection fluid from passing through the connector until sufficient pressure is present in input line to open the normally closed check valve arrangement 2010′. Sufficient fluid pressure to open the check valve arrangement 2010′ or magnetic check valve 2010 may be supplied by the peristaltic pump or other pump mechanism, such as a mechanically or manually operated syringe, and may be in the range of about 8-20 psi.

A check valve arrangement 2010′ may be provided in the connector 1708′ connecting input line with output line on the “high” pressure side of the fluid path set associated with the syringe as shown in U.S. Pat. No. 8,540,698. In this situation, the check valve arrangement 2010′ may be provided in lumen 1778′ in the second connector member 1776′. The locations for the first and second connector members 1774′, 1776′ may be reversed in the connectors 1708′ connecting the respective input lines and output lines.

The check valve assembly 2010′ will generally be discussed as it is situated within the first connector member 1774′ of the connector 1708′ used to connect input line with output line, but the following discussion is equally applicable to the situation where the check valve assembly 2010′ could be associated with the second connector member 1776′. The check valve assembly 2010′ is generally comprised of a retaining sleeve 2012′ and check valve stopper element 2014′. The sleeve 2012′ is disposed (i.e., inserted) within lumen 1777′ and held therein by a friction fit. The lumen 1777′ in the present embodiment of the connector 1708′ includes an extended length conduit receiving cavity 1794′, wherein the sleeve 2012′ is positioned. The conduit receiving cavity 1794′ defines an internal shoulder 2016′. The sleeve 2012′ is disposed within the conduit receiving cavity 1794′ of lumen 1777 so that the sleeve 2012′ abuts the shoulder 2016′. As will be appreciated, flow though the lumen 1777′ will be in the direction of arrow 2018′ when the connector 1708′ is associated with input line. Accordingly, flow through the lumen 1777′ will pass centrally through central bore 2020′ in sleeve 2012′.

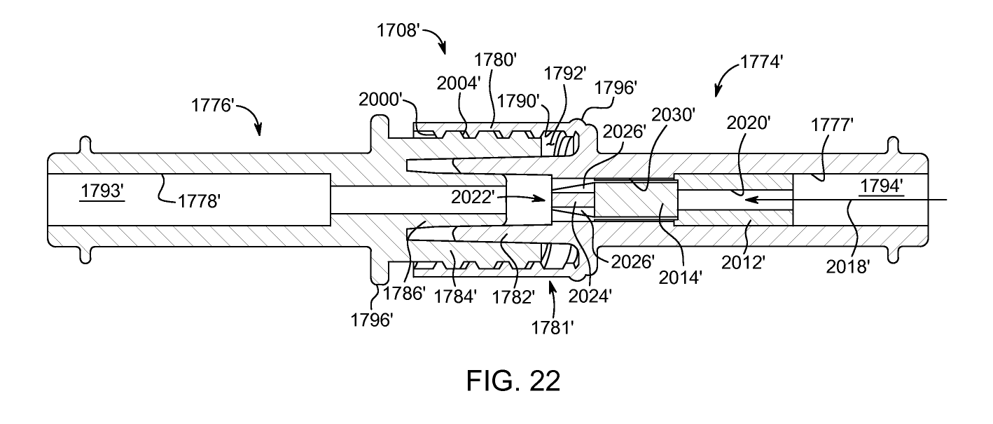

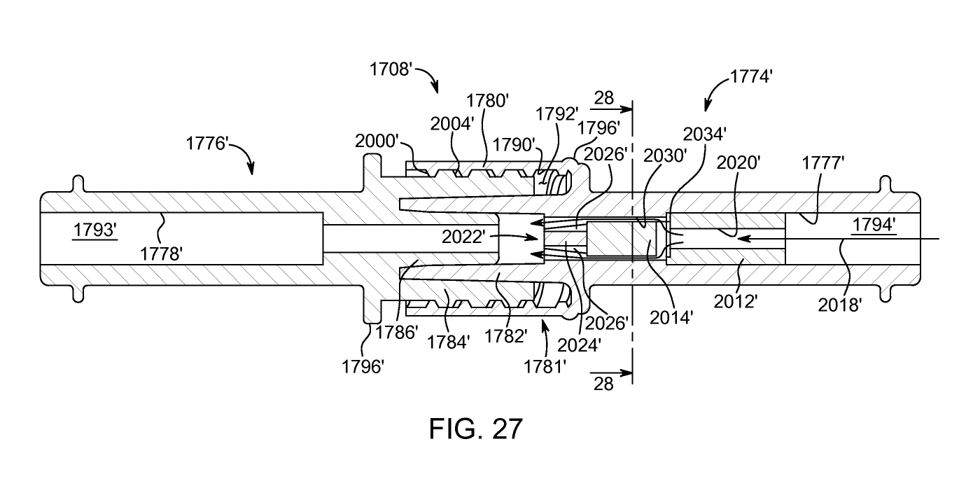

The first luer member 1782′ of the first connector member 1774′ defines a central opening or aperture 2022′ connected to lumen 1777′. The first connector member 1774′ further includes at least one septum 2024′ in the central opening 2022′ which divides the central opening 2022′ into two or more output channels 2026′. The first connector member 1774′ is illustrated in FIGS. 19-28 with only one septum 2024′ for clarity. The septum 2024′ and a distal end 2028′ of the sleeve 2012′ define opposing ends of a cavity 2030′ adapted to receive the stopper element 2014′ (hereinafter “stopper 2014′”). The cavity 2030′ is bounded circumferentially or perimetrically by the wall of lumen 1777′. As shown most clearly in FIG. 21, the second connector member 1776′ may have a similar configuration to the first connector member 1774′ with respect to lumen 1778′ to receive the check valve arrangement 2010′. As shown in FIGS. 22 and 27, the supporting septum 2024′ for the check valve arrangement 2010′ may be omitted from the second connector member 1776′ in the connector 1708′, if desired. The distal end 2028′ of the sleeve 2012′ forms an internal shoulder in lumen 1777′ against which the stopper seats 2014′ to prevent flow through the lumen 1777′ in the normally closed condition of the check valve arrangement 2010′.

In the normally closed condition of the check valve arrangement 2010′, the stopper 2014′ extends between the opposing ends of the cavity 2030′ and seals the central bore 2020′ by engaging the internal shoulder formed by the distal end 2028′ of the sleeve 2012′, thereby preventing flow from passing through the first connector member 1774′ and into the second connector member 1776′. The stopper 2014′ may be formed of a resiliently deformable material such as, a polyethylene thermoplastic elastomer, which deforms when fluid pressure is present in central bore 2020′. The resilient material may be chosen for the stopper 2014′ to have sufficient resiliency to maintain the closure of the central bore 2020′ until a predetermined pressure is reached in the central bore 2020′ and, hence, lumen 1777′. As this predetermined “lift” or deformation pressure is reached, the stopper 2014′ deforms axially a sufficient amount in cavity 2030′ to allow flow to pass from central bore 2020′ into the cavity 2030′. As the stopper 2014′ deforms axially it will unseat from the distal end 2028′ of the sleeve 2012′, thereby allowing flow to exit from the central bore 2020′. As the stopper 2014′ deforms axially, it will simultaneously expand radially. In order to allow fluid to freely pass through cavity 2030′ and into channels 2026′, longitudinal grooves or recesses 2032′ are defined in the wall of cavity 2030′ to permit liquid flow around the stopper 2014′ and through the cavity 2030′. The liquid may then flow through channels 2026′ to enter the second connector member 1776′ and the lumen 1778′ therethrough. Once the fluid pressure is discontinued, for example, by the peristaltic pump shutting-off, the stopper 2014′ will expand axially and again seal against the distal end 2028′ of the sleeve 2012′ to seal the central bore 2020′ and prevent fluid flow through the connector 1708′. The distal end 2028′ may define a circumferential recess 2034′ that will accept the stopper 2014′ to improve the seal between the stopper 2014′ and sleeve 2012′. Since the stopper 2014′ is formed of a resiliently deformable material, the stopper 2014′ may deform or “mold” into this recess 2034′ when the pressure in lumen 1777′ and central bore 2020′ drops to a level sufficient to cause enough axial deformation of the stopper 2014′ to cause the stopper 2014′ to unseat from the distal end 2028′ of the sleeve 2012′.

The foregoing magnetic check valve arrangement 2010 according to the various embodiments described herein has several advantages and improvements over check valves in the prior art including, but not limited to: (1) low and in certain embodiments, variable crack pressure; (2) ability to withstand high fluid pressure; (3) low resistance to fluid flow; (4) a normally closed check valve state due to utilizing magnetic attraction/repulsion for functionality; and (5) magnet(s) may be overmolded into components to provide biocompatibility and particulate protection in the fluid path. The features of the check valve arrangement 2010 may be applied to any of the various embodiments of the connector and connector member in this disclosure or in the disclosure of U.S. Pat. No. 8,540,698.

The foregoing description and accompanying drawings set forth a number of representative embodiments. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A fluid path set for use in a fluid delivery system, the fluid path set comprising:
a connector member defining a lumen for fluid flow through the connector member, and comprising a luer member in fluid connection with the lumen; and
a check valve arrangement disposed in the lumen of the connector member, wherein the check valve arrangement is configured to limit fluid flow to one direction through the connector member, the check valve arrangement comprising:
an overmolded magnetic element disposed in the lumen of the connector member; and
a retaining sleeve disposed in the lumen of the connector member, the retaining sleeve defining a central bore and comprising a distal end wall against which the overmolded magnetic element is adapted to seat to prevent fluid flow through a fluid flow aperture defined in the distal end wall until the overmolded magnetic element is dislodged from the distal end wall.

2. The fluid path set of claim 1, further comprising an end wall magnetic element at the distal end wall of the retaining sleeve, adapted to form a magnetic attractive bond to the overmolded magnetic element.

3. The fluid path set of claim 2, wherein the end wall magnetic element is a magnetically active metal in the distal end wall of the retaining sleeve.

4. The fluid path set of claim 3, wherein the overmolded magnetic element is dislodged from the fluid flow aperture by a pressurized fluid having a crack pressure in the central bore of the retaining sleeve.

5. The fluid path set of claim 2, wherein the end wall magnetic element is an electromagnetic element at the distal end wall of the retaining sleeve, which forms the magnetic attractive bond to the overmolded magnetic element upon application of an electrical current to the electromagnetic element.

6. The fluid path set of claim 5, wherein the electromagnetic element comprises a conductive wire coiled within or around at least one of the distal end wall of the retaining sleeve, a circumferential wall of the retaining sleeve, a circumferential wall of the connector member surrounding at least a portion of the retaining sleeve, a wall of a fluid path retaining element configured for holding the connector, and combinations of any thereof, wherein the coiled conductive wire is in electrical communication with a source of the electrical current.

7. The fluid path set of claim 6, wherein the overmolded magnetic element is dislodged from the fluid flow aperture by at least one of a pressurized fluid having a crack pressure in the central bore of the retaining sleeve, reducing the electrical current applied to the electromagnetic element, reversing the electrical current applied to the electromagnetic element, stopping the electrical current to the electromagnetic element, and combinations thereof.

8. The fluid path set of claim 1, further comprising an arrest disposed in the lumen of the connector member distal to the overmolded magnetic element, wherein the arrest is configured to maintain the overmolded magnetic element in the proximity of the distal end wall of the retaining sleeve when the overmolded magnetic element is dislodged.

9. The fluid path set of claim 8, wherein the arrest comprises an arrest magnetic element oriented to produce a magnetic repulsive force between the arrest magnetic element and the overmolded magnetic element, wherein the magnetic repulsive force forces the overmolded magnetic element to seat against the distal end wall of the retaining element.

10. The fluid path set of claim 9, wherein the arrest magnetic element is a magnetically active metal, wherein a fluid pressure greater than the magnetic repulsive force dislodges the overmolded magnetic element from the distal end wall of the retaining element.

11. The fluid path set of claim 9, wherein the arrest magnetic element is an electromagnetic arrest element in the arrest, which forms the magnetic repulsive force against the overmolded magnetic element upon application of an electrical current to the electromagnetic arrest element.

12. The fluid path set of claim 11, wherein the electromagnetic arrest element comprises a conductive wire coiled within or around at least one of the arrest, a circumferential wall of the connector member surrounding at least a portion of the arrest, a wall of a fluid path retaining element configured for holding the connector, and combinations of any thereof, wherein the coiled conductive wire is in electrical communication with a source of the electrical current.

13. The fluid path set of claim 12, wherein the overmolded magnetic element is dislodged from the fluid flow aperture by at least one of a pressurized fluid having a crack pressure in the central bore of the retaining sleeve greater than the magnetic repulsive force, reducing the electrical current applied to the electromagnetic arrest element, reversing the electrical current applied to the electromagnetic arrest element, stopping the electrical current to the electromagnetic arrest element, and combinations thereof.

14. The fluid path set of claim 1, wherein the overmolded magnetic element is cylindrical, ellipsoidal, or spherical in shape

15. A connector for a fluid path set, the connector comprising:
a connector member defining a lumen for fluid flow through the connector member; and
a magnetic check valve arrangement disposed in the lumen of the connector member, wherein the magnetic check valve arrangement is configured to limit fluid flow to one direction through the connector member, the magnetic check valve arrangement comprising:
an overmolded magnetic element disposed in the lumen of the connector member; and
a retaining sleeve disposed in the lumen of the connector member, the retaining sleeve defining a central bore and comprising a distal end wall against which the overmolded magnetic element is adapted to seat to prevent fluid flow through a fluid flow aperture defined in the distal end wall until the overmolded magnetic element is dislodged from the distal end wall.

16. The connector of claim 15, further comprising an end wall magnetic element at the distal end wall of the retaining sleeve, adapted to form a magnetic attractive bond to the overmolded magnetic element.

17. The connector of claim 16, wherein the end wall magnetic element is a magnetically active metal in the distal end wall of the retaining sleeve.

18. The connector of claim 16, wherein the end wall magnetic element is an electromagnetic element at the distal end wall of the retaining sleeve, which forms the magnetic attractive bond to the overmolded magnetic element upon application of an electrical current to the electromagnetic element.

19. A method for reversibly sealing a valve of a fluid delivery system reactive to a specified pressure, the method comprising:
forming a magnetic attractive bond between an overmolded magnetic element and a distal end wall of a retaining sleeve disposed within a lumen,
wherein the overmolded magnetic element is seated over and prevents fluid flow through a fluid flow aperture defined in the distal end wall and wherein the magnetic attractive bond has a magnetic attractive bond strength equal to a specified pressure of a fluid within the lumen.

20. The method of claim 19, further comprising
flowing a pressurized fluid through the lumen, wherein the pressurized fluid has a pressure greater than or equal to the specified pressure; and
dislodging the overmolded magnetic element from the distal end wall, thereby allowing fluid flow through the fluid flow aperture.
